(19) United States
(12) Patent Application Publication
(10) Pub. No.: US 2015/0141850 A1
(43) Pub. Date: May 21, 2015

(54) BALLOON CATHETER FOR MEASURING THE LENGTH OF A STENOSIS

(71) Applicant: ASSISTANCE PUBLIQUE-HOPITAUX PARIS, Paria (FR)

(72) Inventor: Alain Attar, Paris (FR)

(73) Assignee: ASSISTANCE PUBLIQUE- HOPITAUX DE PARIS, Paris (FR)

(21) Appl. No.: 14/407,766

(22) PCT Filed: Jun. 12, 2013
(86) PCT No.: PCT/EP2013/062170

§ 371 Date: Dec. 12, 2014

Related U.S. Application Data
(60) Provisional application No. 61/659,247, filed on Jun. 13, 2012.

(51) Int. Cl. A61B 5/107; A61M 25/10; A61B 1/018; A61B 1/04; A61B 5/02; A61B 5/00

(52) U.S. Cl. CPC A61B 5/1076; A61B 5/02007; A61B 5/6853; A61B 1/018; A61B 1/04; A61M 25/10; A61M 2025/0008
USPC 600/481

(57) ABSTRACT

A balloon catheter adapted for cooperating with an endoscope, the balloon catheter including a catheter shaft, a balloon located at a catheter shaft tip, wherein the catheter shaft includes visual markers designed for determining the length of a stenosis in a bodily structure.

### FIELD OF THE INVENTION

The present invention relates to the field of catheters employed in the observation, diagnosis and treatment of vessels, body cavities and the like. More particularly, the invention relates to a balloon catheter adapted for measuring the length of a stenosis, in cooperation with endoscopy means.

### BACKGROUND

A stenosis is an abnormal narrowing in a blood vessel or other tubular organ or structure. A stenosis can occur because of an inflammation, for example in an intestine because of chronic inflammations due to the Crohn's disease.

A drastic treatment of a stenosis is chirurgical resection. However, if the stenosis is situated on an accessible zone to endoscopy, it can be treated by dilatation as an alternative to chirurgical resection. Dilatation is performed by means of an endoscope coupled with a balloon catheter. An endoscope is a medical device used to examine the interior surfaces of an organ or tissue, consisting of a long and thin tube which has a light and a video camera located at its end. Images of the inside of the patient's body can be seen on a screen. A balloon catheter is a soft catheter with an inflatable balloon at its tip. The balloon is either circular or oblong, and can be inflated via air (pneumatic dilatation) or via water (hydrostatic dilatation). During a dilatation procedure, the endoscope is introduced into a strictured bodily structure, and aims at locating the stenosis. If the stenosis has a diameter smaller than the one of the endoscope, the endoscope is stopped in front of the stenosis. The balloon catheter is introduced through an operator canal of the endoscope, and then through the stenosis. Finally, the balloon is positioned into the stenosis and progressively inflated, so as to dilate the lumen of the bodily structure to enlarge the stenosis. The operation is repeated if required. This simple method has great success rates, but can be inefficient if the stenosis is too long: indeed, dilatations have to be performed little by little along the stenosis, which is technically difficult to perform and unpredictable as for the effective dilatation of the stenosis. Therefore, measuring the length of the stenosis prior to a dilatation intervention is crucial for choosing the best appropriate shape and dimensions of the balloon.

Another treatment is a stent placement so as to dilate the stenosis in a sustained way, during a long time. Measuring the length of the stenosis is also necessary so as to choose a well-suited stent.

For now, measuring the length of a stenosis is a rough estimate, and dimensions of the balloon catheter or the stent are often chosen upward.

### SUMMARY

It is an object of the invention to provide a device for precisely measuring the length of a stenosis.

To this end, there is provided a balloon catheter adapted for cooperating with an endoscope, said balloon catheter comprising:
a catheter shaft,
a balloon located at a catheter shaft tip,
wherein said catheter shaft comprises visual markers designed for determining the length of a stenosis in a bodily structure.

According to not limited embodiments, the balloon catheter can comprise one or more of the following additional characteristics:
the balloon catheter is adapted for passing through an operator canal of the endoscope;
visual markers are adapted for being read by a camera of the endoscope;
visual markers extend from the catheter shaft tip along the catheter shaft;
visual markers are centimeter graduations;
visual markers are coloured sections;
visual markers extend on the whole periphery of the catheter shaft.

In addition, there is provided a device for measuring the length of a stenosis in a bodily structure, comprising:
an endoscope,
a balloon catheter as previously described, adapted for cooperating with said endoscope.

In addition, there is provided a method for measuring the length of a stenosis in a bodily structure, comprising:
Introducing an endoscope into said bodily structure until reaching the beginning of the stenosis;
Passing a balloon catheter as previously described through an operator canal of the endoscope, until the balloon comes out of the operator canal and crosses widely the stenosis;
Inflating the balloon until the balloon has dimensions wider than dimensions of the stenosis;
Pulling the balloon catheter back, until the balloon is stucked by the stenosis;
Reading the visual marker on the catheter shaft, that is closest of a camera of the endoscope.

According to a not limited embodiment, the method can comprise rotating the catheter shaft so that the camera can read the visual marker.

### BRIEF DESCRIPTION OF THE DRAWING

An embodiment of a balloon catheter in accordance with the present invention is now described, by way of example only, and with reference to the accompanying drawings, in which:

FIG. 1 schematically illustrates a balloon catheter introduced a strictured bodily structure, in accordance with the present invention;

FIG. 2 is a flow chart of a not limited embodiment of a method in accordance with the present invention.

### DESCRIPTION OF EMBODIMENTS

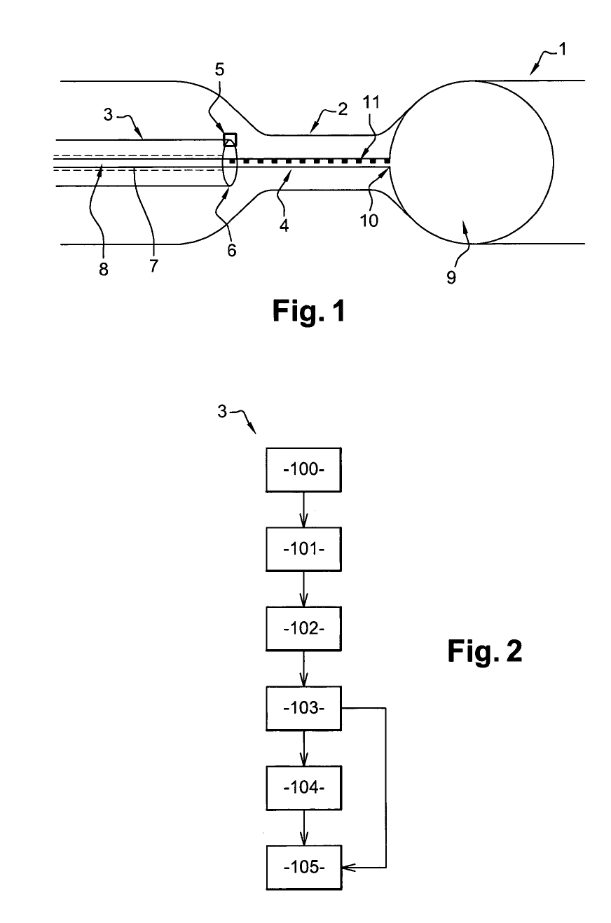

FIG. 1 schematically illustrates a portion of a colon 1 comprising a stenosis 2, in which an endoscope 3 and a balloon catheter 4 are located.

The endoscope 3, well-known by the man skilled of the art, comprises:
a video camera 5 located at one end 6 of the endoscope 3. The video camera 5 is used for transmitting images of the colon 1 to a screen.
an operator canal 7 extending along the endoscope 3. The operator canal 7 is classically adapted for receiving a catheter.

The balloon catheter 4 comprises:
a cylindrical catheter shaft 8;
an inflatable balloon 9 situated at one tip 10 of the catheter shaft 8. In this not limited embodiment, the balloon 9 is circular and can be inflated via air.

In other not represented embodiments, the balloon is oblong, or can be inflated by water, or is inflated with or without contrast agent.
visual markers 11 extending from the tip 10 along the catheter shaft 8. In this not limited embodiment, the visual markers 11 are centimeter graduations, but in another embodiment, the visual markers are coloured sections: each colour is associated with a length. Indeed, if the colon 1 is not perfectly clean, it can be difficult to read centimeter graduations 11, which is easier with some coloured sections. It is to be noted that all visual markers designed to determine a length can be used. Besides, numbers can be inscribed on the catheter shaft so as to help the user to read a graduation.

The balloon catheter 4 cooperates with the endoscope 3:
in the represented position, the catheter shaft 8 extends partly along the operator canal 7, and
the visual markers 11 are designed to be read by the video camera 5.

As represented in FIG. 1, the balloon 9 is inflated and stucked downstream of the colon 1 by the stenosis 2. Indeed, the diameter of the balloon 9 is wider than the diameter of the stenosis 2. It is to be noted that “upstream” and “downstream” are defined with respect to the moving of the endoscope 3 or the balloon catheter 4 when they are introduced in the colon 1. For example, if the endoscope 3 and the balloon catheter 4 are introduced in the opposite direction of the peristalsis bowel movements, “upstream” represents the patient's anus side. The device according to the invention may also be used in the peristalsis direction.

As represented in FIG. 1, the diameter of the balloon 9 is adapted to the diameter of the colon 1. For instance, for measuring a colonic stenosis, the balloon may be inflated larger than 30 millimeters, but for measuring oesophageal or small intestine stenosis, the balloon may be inflated less than 20 millimeters.

The association of the endoscope 3 and the balloon catheter 4 constitute a device for measuring the length of the stenosis 2, and a measuring method 120 is described hereafter and schematically illustrated in FIG. 2. The method 120 comprises:
Introducing 100 the endoscope 3 into the colon 1, until reaching the upstream beginning of the stenosis 2. The stenosis 2 is localizable thanks to the video camera 5.
Passing 101 the non-inflated balloon catheter 4 through the operator canal 7 of the endoscope 3, until the balloon 9 comes out of the operator canal 7 and crosses widely the stenosis 2: in this position, the balloon 9 is downstream of the stenosis 2.
Inflating 102 the balloon 9 until the balloon 9 has dimensions wider than dimensions of the stenosis 2.
Pulling 103 the balloon catheter 4 upstream, until the balloon 9 is stucked by the stenosis 2. The pulling has to be a soft and constant traction.
Reading 105 the centimetre graduation 11 on the catheter shaft 8, that is closest of the video camera 5. In the represented example in FIG. 1, the operator can estimate that the stenosis 2 has a length which is about 10 centimeters. If the video camera 5 can not read the visual marker 11 because it is not well-oriented, the operator has to rotate 104 the balloon catheter 4. This can be avoided by circular visual markers 11 which extend on the whole periphery of the catheter shaft 8.

It is to be noted that a huge advantage of the balloon catheter according to the invention is that is can be used for pneumatic or hydrostatic dilatation, after the measure of the stenosis, which is convenient for fabrication and use.

The description and drawing merely illustrate the principles of the invention. It will thus be appreciated that those skilled in the art will be able to devise various arrangements that, although not explicitly described or shown herein, embody the principles of the invention and are included within its spirit and scope. Furthermore, all examples recited herein are principally intended expressly to be only for pedagogical purposes to aid the reader in understanding the principles of the invention and the concepts contributed by the inventor to furthering the art, and are to be construed as being without limitation to such specifically recited examples and conditions. Moreover, all statements herein reciting principles, aspects, and embodiments of the invention, as well as specific examples thereof, are intended to encompass equivalents thereof.

1. A balloon catheter adapted for cooperating with an endoscope, said balloon catheter comprising:
a catheter shaft, and
a balloon located at a catheter shaft tip,
wherein said catheter shaft comprises visual markers designed for determining the length of a stenosis in a bodily structure.

2. The balloon catheter according to claim 1, adapted for passing through an operator canal of the endoscope.

3. The balloon catheter according to claim 1, wherein visual markers are adapted for being read by a camera of the endoscope.

4. The balloon catheter according to claim 1, wherein visual markers extend from the catheter shaft tip along the catheter shaft.

5 The balloon catheter according to claim 1, wherein visual markers are centimeter graduations.

6 The balloon catheter according to claim 1, wherein visual markers are coloured sections.

7 The balloon catheter according to claim 1, wherein visual markers extends on the whole periphery of the catheter shaft.

8. A device for measuring the length of a stenosis in a bodily structure, comprising
an endoscope, and
a balloon catheter according to claim 1, adapted for cooperating with said endoscope.

9. A method for measuring the length of a stenosis in a bodily structure, comprising:
introducing an endoscope into said bodily structure until reaching the beginning of the stenosis,
passing a balloon catheter according to claim 1 through an operator canal of the endoscope, until the balloon comes out of the operator canal and crosses widely the stenosis,
inflating the balloon until the balloon has dimensions wider than dimensions of the stenosis,
pulling the balloon catheter back, until the balloon is stucked by the stenosis, and
reading the visual marker on the catheter shaft, that is closest of a camera of the endoscope.

10. The method according to claim 9, comprising rotating the catheter shaft so that the camera can read the visual marker.
